(19) United States
(12) Patent Application Publication
(10) Pub. No.: US 2012/0078014 A1
(43) Pub. Date: Mar. 29, 2012

(54) METHOD FOR MANUFACTURING CYCLOHEXANONE OXIME

(75) Inventors: Masami FUKAO, Niihama-shi (JP); Hiroshi TOMOI, Niihama-shi (JP)

(73) Assignee: SUMITOMO CHEMICAL COMPANY, LIMITED, Tokyo (JP)

(21) Appl. No.: 13/240,532

(22) Filed: Sep. 22, 2011

(30) Foreign Application Priority Data
Sep. 27, 2010 (JP) 2010-214835

(51) Int. Cl. C07C 249/04

(52) U.S. Cl. 564/267

(57) ABSTRACT

To produce cyclohexanone oxime stably for a long time by an ammoximation reaction of cyclohexanone. Cyclohexanone oxime is produced by performing an ammoximation reaction of cyclohexanone with hydrogen peroxide and ammonia in the presence of titanosilicate and a solid containing a silicon compound, wherein the solid containing a silicon compound is one that had been used in a Beckmann rearrangement reaction of cyclohexanone oxime as a catalyst.

### TECHNICAL FIELD

The present invention relates to a method for manufacturing cyclohexanone oxime by ammoximation of cyclohexanone.

### BACKGROUND

As a method for producing cyclohexanone oximes, an ammoximation reaction of cyclohexanone with hydrogen peroxide and ammonia using titanosilicate as a catalyst is known (e.g., Patent documents 1-4). Generally in this ammoximation reaction, since the catalytic activity of titanosilicate gradually declines, it is required to renew titanosilicate in order to maintain a conversion to cyclohexanone over a predetermined value. When the frequency of renewal is high, it may cause a problem in cost performance of catalyst. Therefore, as a method for suppressing decline in a catalytic activity of titanosilicate in the ammoximation reaction, for example, proposed are a method for performing the above-mentioned reaction by placing titanosilicate and amorphous silica such as silica gel and fumed silica together (Patent Documents 5 and 6) and a method for performing the above-mentioned reaction by placing fresh titanosilicate and titanosilicate used in the above-mentioned reaction together (Patent Document 7).

### BACKGROUND ART

### Patent Documents

### [Patent Document 1] JP S62-59256A

### [Patent Document 2] JP S63-130575A

### [Patent Document 3] JP HP06-49015A

### [Patent Document 4] JP H06-92922A

### [Patent Document 5] JP 2004-83560A

### [Patent Document 6] JP 2007-182428A

### [Patent Document 7] JP 2004-307418A

### DISCLOSURE OF THE INVENTION

### Problem to be Solved by the Invention

However, in the above-mentioned ammoximation reaction, it is difficult to thoroughly suppress decline in the catalytic activity of titanosilicate and the reaction is not satisfactory in a catalytic lifetime because a catalyst gradually degrades in a time course of reaction. Therefore, an object of the present invention is to provide a method capable to manufacture cyclohexanone oxime stably for a long time.

### Means for Solving the Problem

The present inventors excessively studied to solve the problem and found that the object of the present invention can be achieved by performing an ammoximation reaction of cyclohexanone in the presence of a solid containing a silicon compound and titanosilicate, wherein the solid containing a silicon compound had been used in a Beckmann rearrangement reaction of cyclohexanone oxime as a solid catalyst, to accomplish the present invention.

More specifically, the present invention provides:

[1] A method for manufacturing cyclohexanone oxime comprising

performing an ammoximation reaction of cyclohexanone with hydrogen peroxide and ammonia in the presence of titanosilicate and a solid containing a silicon compound, wherein the solid containing a silicon compound is that had been used as a catalyst in a Beckmann rearrangement reaction of cyclohexanone oxime;

[2] The method according to [1], wherein the silicon compound is at least one selected from a group consisting of zeolite, silica alumina, a complex oxide of silica and a metal oxide other than silica, and amorphous silica;
[3] The method according to [1] or [2], wherein the solid further contains cokes;
[4] The method according to [3], wherein a carbon content is 5.0 weight % or lower in the solid; and
[5] The method according to [3], wherein a carbon content is 5.0 weight % or lower and a nitrogen content is 0.50 weight % or lower in the solid.

### Effects of the Invention

According to the present invention, cyclohexanone oxime can be manufactured stably for a long time with suppressing decline in a catalytic activity of titanosilicate.

### MODE FOR CARRYING OUT THE INVENTION

The present invention will be explained in detail below. The present invention uses cyclohexanone as a raw material and an ammoximation reaction of cyclohexanone is performed with hydrogen peroxide and ammonia in the presence of titanosilicate and a solid containing a silicon compound to produce cyclohexanone oxime.

The raw material cyclohexanone may be obtained by, for, example, oxidation of cyclohexane, dehydrogenation of cyclohexanol, hydration and dehydrogenation of cyclohexene or hydrogenation of phenol.

The amount of hydrogen peroxide to be used is generally 0.5-3.0 mole equivalents, preferably 0.5-1.5 mole equivalents with respect to cyclohexanone. Hydrogen peroxide is usually manufactured by an anthraquinone method and commercially sold as an aqueous solution generally at 10-70 weight %. This may be used. In addition, a stabilizer, for example, phosphate such as sodium phosphate; polyphosphate such as sodium pyrophosphate and sodium tripolyphosphate; pyrophosphoric acid; ascorbic acid; ethylenediaminetetraacetic acid; diethylenetriaminepentaacetic acid and the like may be added to hydrogen peroxide.

Ammonia may be used in a gaseous state or a liquid state, or may be used as a solution in water or an organic solvent. The amount of ammonia to be used is preferably 1.0 mole or higher, more preferably 1.5 moles or higher with respect to 1 mole of cyclohexanone. Further, the amount of ammonia to be used is preferably excessive over hydrogen peroxide so as to remain in a reaction mixture. In addition, the concentration of ammonia in a liquid phase of the reaction mixture is preferably adjusted to 1 weight % or higher. As above, the concentration of ammonia in the liquid phase of the reaction mixture is adjusted at a predetermined value of higher to enhance the conversion of cyclohexanone and the selectivity of cyclohexanone oxime and, in turn, the yield of cyclohexanone oxime is also enhanced. This concentration of ammonium is preferably 1.5 weight % or higher and, usually 10 weight % or lower, preferably 5 weight % or lower.

The ammoximation reaction according to the present invention is preferably performed by using water and/or an organic solvent as a reaction medium. Examples of the organic solvent include alcohols, aromatic hydrocarbons and ethers and optionally two or more kinds of them may be used together. Among them, alcohols are preferable. Preferable alcohols are alcohols having a carbon number of 1-6, for example, methyl alcohol, ethyl alcohol, n-propyl alcohol, isopropyl alcohol, n-butyl alcohol, s-butyl alcohol, t-butyl alcohol and t-amyl alcohol and optionally two or more kinds of them may be used together. Preferable aromatic hydrocarbons are, for example, benzene, toluene, xylene and ethyl benzene and optionally two or more kinds of them may be used together. Preferable ethers are, for example, tetrahydrofuran, dioxane, diisopropyl ether, t-butyl methyl ether and optionally two or more kinds of them may be used together. The amount of the reaction medium to be used is usually 0.2-10 parts by weight, preferably 1-5 parts by weight with respect to 1 part by weight of cyclohexanone.

The ammoximation reaction according to the present invention uses titanosilicate as a catalyst. This titanosilicate comprises titanium, silicon and oxygen as network elements and it may be that has a network substantially consisting of titanium, silicon and oxygen and may further comprise elements other than titanium, silicon and oxygen as network elements as far as they do not interfere the ammoximation reaction. Embodiments of titanosilicate include Ti-MWW represented by Ti-MCM-22 which is crystalline titanosilicate having a MWW structure; TS-1 which is crystalline titanosilicate having a MFI structure; TS-2 which is crystalline titanosilicate having a MEL structure; Ti-MCM-41 which is non-crystalline titanosilicate having a mesopore structure, and the like. In this context, MWW, MFI and MEL are framework type codes for zeolite defined by International Zeolite Association (IZA). Titanosilicate having an atom ratio of silicon/titanium of 10-1000 are used suitably. The form may be fine powder, optionally shaped into granules or pellets using a binder or may be supported on a matrix. The particle size is preferably 0.001-1.0 mm, more preferably 0.005-0.20 mm.

In the present invention, the ammoximation reaction is performed in the presence of titanosilicate as well as a solid containing a silicon compound, wherein the solid containing a silicon compound had been used in a Beckmann rearrangement reaction of cyclohexanone oxime as a catalyst (hereinafter, a “solid containing silicon compound which had been used in a Beckmann rearrangement reaction of cyclohexanone oxime as a catalyst” may be referred to as simply a “solid containing a silicon compound”). This method can suppress decline in a catalytic activity of titanosilicate and, in turn, renewal frequency of titanosilicate can be decreased to reduce a cost for a catalyst. Further, the amount of titanosilicate to be placed in an ammoximation reaction system can also be decreased and, therefore, a cost for a catalyst can be reduced. Additionally, this solid containing a silicon compound to be used by itself does not have substantially an activity as a catalyst for an ammoximation reaction.

It is presumed that the decline in the catalytic activity of titanosilicate is caused due to a collapse of active sites by elution of silicon and an attenuation of active sites by adsorption of impurities. It is presumed that the decline in the catalytic activity of titanosilicate is suppressed by co-existence of the solid containing a silicon compound because silicon elutes from the silicon compound to suppress elution of silicon from titanosilicate and because impurities are adsorbed on the solid containing a silicon compound to suppress adsorption of impurities on titanosilicate. Therefore, solids containing a silicon compound in which silicon easily elutes or impurities are easily adsorbed in the ammoximation reaction are used preferably and further those having a large surface area or a small particle size are used more preferably. From this viewpoint, solids containing a silicon compound which had been used in a Beckmann rearrangement reaction of cyclohexanone oxime are used because such solids are considered to contain a silicon compound as a silicon source for the ammoximation reaction and to easily adsorb organic compounds which are impurities on a catalyst.

The silicon compounds are, not limited as far as they can be used in a Beckmann rearrangement reaction of cyclohexanone oxime as a solid catalyst, preferably contain silicon and oxygen. Specifically, zeolite, silica alumina, a complex oxide of silica and a metal oxide other than silica, amorphous silica, and the like are used and, optionally two or more kinds of them may be used together. Among them, zeolites such as crystalline silica, crystalline aluminosilicate, crystalline metallosilicate, and the like are preferable. Among these zeolites, zeolites having a pentasil-type structure, zeolites having a Y-type structure, zeolites having a β-type structure, zeolites having a L-type structure, zeolites having a mordenite-type structure are preferable and zeolites having a pentasil-type structure are more preferable. Among zeolites having a pentasil-type structure, zeolites having a MFI structure are particularly preferable.

The zeolite is that has a network comprising silicon and oxygen as network elements and, it may be crystalline silica having a network substantially consisting of silicon and oxygen, a crystalline metallosilicate comprising other elements as network elements as far as they do not interfere the ammoximation reaction, or the like. Among them, crystalline silica having a network consisting of silicon and oxygen and having a MFI structure (Silicalite-1) is preferable. The primary particle size of the zeolites is preferably 5 μm or smaller, more preferably 1 μm or smaller.

The zeolite may be suitably prepared, for example, by performing hydrothermal synthesis of a raw material silicon compound which is a raw material for zeolite, a quaternary ammonium compound, water and, optionally a metallic compound, drying and firing the resultant crystals followed by contact treatment with ammonia or an ammonium salt, and by drying it.

The form of the silicon compound used in the Beckmann rearrangement reaction as a catalyst may be a powder of a silicon compound, a molded product substantially comprising only a silicon compound, a molded product comprising a mixture of a silicon compound and a binder or reinforcing material or a silicon compound supported on a matrix. The particle size is preferably 0.001-5 mm, more preferably 0.01-3 mm.

The Beckmann rearrangement reaction may be performed in a manner of fixed bed, fluidized bed or moving bed preferably under a gas phase condition to produce ε-caprolactam. The reaction temperature is usually 250-500° C., preferably 300-450° C. The reaction pressure is usually 0.005-0.5 MPa, preferably 0.005-0.2 MPa as an absolute pressure.

As stated above, when the Beckmann rearrangement reaction of cyclohexanone oxime is performed in the presence of the silicon compound under a gas phase condition, usually, as a reaction time passes, in other words, as an accumulated throughput of cyclohexanone oxime per unit weight of a catalyst increases, so-called cokes are gradually adsorbed on the silicon compound due to condensation or polymerization of cyclohexanone oxime, ε-caprolactam, byproducts and the like. As a result, the catalytic activity gradually declines, that is, the conversion of cyclohexanone oxime. Therefore, in order to recover the catalytic activity in the Beckmann rearrangement reaction by removing cokes from a coke-adhered silicon compound, a Beckmann rearrangement reaction catalyst-regeneration step is generally provided, which comprises a heat treatment in an atmosphere of an oxygen-containing gas.

As for the oxygen-containing gas used in the Beckmann rearrangement reaction catalyst-regeneration step, an air is usually suitable but an air or oxygen may be diluted with an inert gas such as nitrogen, argon and carbon dioxide to use. The oxygen concentration in the oxygen-containing gas is usually 1-30 volume %, preferably 5-25 volume %. The heat treatment temperature in the Beckmann rearrangement reaction catalyst-regeneration step is usually 200° C.-600° C., preferably 200° C.-450° C. The silicon compound catalysis treated in the Beckmann rearrangement reaction catalyst-regeneration step may be recycled as a catalyst in the Beckmann rearrangement reaction.

The solid containing a silicon compound according to the present invention is not particularly limited as far as those used in the Beckmann rearrangement reaction of cyclohexanone oxime as a solid catalyst, may be a solid containing a silicon compound and cokes, which is obtained in the Beckmann rearrangement reaction step or a solid containing a silicon compound in which the cokes are eliminated, which is obtained in a Beckmann rearrangement reaction catalyst-regeneration step. Additionally, in the solid containing a silicon compound in which the cokes are eliminated in the Beckmann rearrangement reaction catalyst-regeneration step, it is not essential to eliminate the cokes thoroughly and it is accepted that cokes remains. Further, the solid containing a silicon compound may be those removed from a Beckmann rearrangement reaction step or a Beckmann rearrangement reaction catalyst-regeneration step during cycling operations of Beckmann rearrangement reaction steps and Beckmann rearrangement reaction catalyst-regeneration steps or catalysts removed from a Beckmann rearrangement reaction step or a Beckmann rearrangement reaction catalyst-regeneration step after suspension of operations because the catalyst loses a desired performance due to deposition of cokes on the catalyst or thermal decomposition of the catalyst in a course of an oration time or a period for usage of a catalyst. Further, catalysts removed from a Beckmann rearrangement reaction step or a Beckmann rearrangement reaction catalyst-regeneration step during or after operations may be separately activated for recovering an activity and selectivity for the Beckmann rearrangement reaction to use. In the method for producing cyclohexanone oxime according to the present invention, as a solid containing a silicon compound, a solid containing a used silicon compound may be used, a so-called spent catalyst which will be disposed because a desired performance for a Beckmann rearrangement reaction is lost due to a long-time use. This is industrially useful from viewpoints of environmental protection and cost reduction through reduction and effective utilization of waste materials.

When the solid containing a silicon compound further comprises cokes, the solid will contain carbon components derived from the cokes and may contain nitrogen components derived from the cokes in addition to the carbon components. The carbon content in the solid containing a silicon compound and cokes is preferably 5.0 weight % or lower, more preferably 0.01-4.0 weight %. When the solid containing a silicon compound and cokes comprises a nitrogen component, the nitrogen content in the solid is preferably 0.50 weight % or lower, more preferably 0.001-0.40 weight %.

The carbon content and the nitrogen content in the solid containing a silicon compound are obtained by a total carbon (TC) measurement and a total nitrogen (TN) measurement, respectively, for a solid catalyst. Specifically, for example, amounts of carbon oxides and nitrogen oxides generated by oxygenizing a predetermined amount of the solid with a oxygen gas are measured through gas chromatography, infrared spectroscopy and the like and the amount of carbon oxides and the amount of nitrogen oxides are converted into an amount of carbon atoms and an amount of nitrogen atoms, respectively, and each of them are divided by the amount of the solid to obtain the carbon content and the nitrogen content.

Since both the carbon content and the nitrogen content in the solid containing a silicon compound usually increase in a Beckmann rearrangement reaction step and decrease in a Beckmann rearrangement reaction catalyst-regeneration step, the carbon content and the nitrogen content in the solid containing a silicon compound may be maintained within the above ranges by adjusting a supply amount of cyclohexanone oxime or conditions such as a reaction time (a retention time) in the reaction step to prevent overdeposition of coke components on the solid containing a silicon compound or by adjusting conditions such as a thermal treatment temperature and a thermal treatment time (a retention time) in the catalysis-regeneration step to eliminate coke components depositing of the solid containing silicon compound.

Thus obtained solid containing a silicon compound used in a Beckmann rearrangement reaction of cyclohexanone oxime as a catalyst is placed together with titanosilicate in the ammoximation reaction. This ammoximation reaction may be performed as a solid catalytic reaction in which the solid containing a silicon compound and titanosilicate disperse in the reaction mixture. The amount of titanosilicate to be placed in the ammoximation reaction system is usually 1-200 g/L as a weight per volume of the reaction mixture (a solid phase and a liquid phase). The amount of the solid containing a silicon compound to be placed in the ammoximation reaction system is preferably 0.1-20 weight equivalent with respect to titanosilicate.

The ammoximation reaction may be performed in a batch or continuous manner and it is better to perform it in a continuous manner from viewpoints of productivity and operability. A means for introducing raw materials may be suitably selected. In a batch manner, for example, the reaction may be performed by placing cyclohexanone, ammonia, titanosilicate, solid containing a silicon compound and a solvent in a reactor followed by supplying hydrogen peroxide hereto; by placing cyclohexanone, titanosilicate, solid containing a silicon compound and a solvent in a reactor followed by supplying hydrogen peroxide and ammonia; or by placing titanosilicate, solid containing a silicon compound and a solvent in a reactor followed by supplying cyclohexanone, hydrogen peroxide and ammonia. Additionally, when the ammoximation reaction is performed in a batch manner, the solid containing a silicon compound and/or titanosilicate may be added during the reaction.

In a continuous manner, the reaction is performed by retaining a predetermined amount of a reaction mixture in which a solid containing a silicon compound and titanosilicate are dispersed within a reactor with supplying cyclohexanone, hydrogen peroxide, ammonia and a solvent and by removing the reaction mixture approximately at the same amount of these raw materials, wherein the reaction mixture is removed through a filter and the like to remove only a liquid phase so as to leave a solid phase of the solid containing a silicon compound and titanosilicate within the reactor. In the ammoximation reaction in a continuous matter, the solid containing a silicon compound and/or titanosilicate may be added continuously or intermittently into the reaction system. When the solid containing a silicon compound and/or titanosilicate are added, the solid containing a silicon compound and/or titanosilicate may be appropriately removed for maintaining a good mixing state where the solid containing a silicon compound and titanosilicate are uniformly dispersing as well as the catalytic activity. Additionally, reactors lined with fluorine resin or glass or of stainless steels are preferably used from a viewpoint of preventing decomposition of hydrogen peroxide.

The reaction temperature for the ammoximation reaction is preferably 60° C. or higher, more preferably 80° C. or higher, further preferably 90° C. or higher and, preferably 120° C. or lower, more preferably 110° C. or lower, further preferably 100° C. or lower. The reaction pressure for the ammoximation reaction may be either under a normal pressure, an increased pressure and a reduced pressure, it is preferable to perform the reaction under an increased pressure in order to enhance solubility of ammonia into a reaction mixture and then the pressure may be adjusted by using an inert gas such as nitrogen and helium. When the ammoximation reaction is performed under an increased pressure, the reaction pressure is preferably 0.05-1.0 MPa, more preferably 0.1-0.5 MPa as an absolute pressure.

In the ammoximation reaction, for example, a remaining concentration of cyclohexanone, a remaining concentration of hydrogen peroxide or an amount of a byproduct such as oxygen in the reaction mixture are used as indicators for decline of the catalytic activity. Specifically, a cyclohexanone conversion obtained from the remaining concentration of cyclohexanone calculated by gas chromatography analysis of a liquid phase of the reaction mixture or an oxygen concentration in an exhaust gas obtained by analysis of gas exhausted from the reactor with introducing an inert gas such as nitrogen or helium into the reactor are used as indicators. The addition or removal of the solid containing a silicon compound and/or titanosilicate may be performed so as to keep the cyclohexanone conversion at or above the predetermined value or keep the oxygen concentration at or below the predetermined value. Conditions such as a reaction temperature and a reaction pressure in the ammoximation reaction may be adjusted to keep the cyclohexanone conversion at or above the predetermined value or to keep the oxygen concentration at or below the predetermined value.

Titanosilicate used in an ammoximation reaction may be activated to reuse in another ammoximation reaction. By this, a cost for catalysis can be further reduced.

The activation of titanosilicate used in an ammoximation reaction may be performed by firing in an atmosphere of an oxygen-containing gas, and it is preferable to fire particularly under a stream of an oxygen-containing gas. An air is usually used as an oxygen-containing gas and pure oxygen may be also used. These may be used with diluting an inert gas such as nitrogen, carbon dioxide, helium and argon and then its oxygen concentration is 5 volume % or more. Since when the firing temperature is so low, it takes a long time to fire, it is usually 250° C. or higher, preferably 300° C. or higher and usually 600° C. or lower, preferably 550° C. or lower. The firing time may be appropriately adjusted based on the firing temperature and the like, it is usually about 5 minutes to 48 hours, preferably 3-24 hours. Additionally, the firing pressure is arbitrally but usually a normal pressure.

Titanosilicate used in an ammoximation reaction is usually in a state where it is mixed with the solid containing a silicon compound, titanosilicate may be fired together with the solid containing a silicon compound in the mixed state. It may be optionally washed with water or an organic solvent or pre-dried before firing. The pre-drying is performed preferably at 80-150° C.

Firing may be performed in a batch or continuous manner. In a batch manner, the reaction is performed by placing predetermined amounts of the solid containing a silicon compound and titanosilicate used in the ammoximation reaction in a firing furnace such as an oven followed by introducing an oxygen-containing gas. In a continuous manner, the reaction is performed by introducing an oxygen-containing gas into a firing furnace such as a kirn, by introducing a solid containing a silicon compound and titanosilicate used in an ammoximation reaction at a predetermined speed and retaining them for a predetermined period followed by removing them.

In this manner, a solid containing a silicon compound and titanosilicate used in the ammoximation reaction may be recycled by fired to reuse in the ammoximation reaction (hereinafter, a “solid containing silicon compound obtained by firing solid containing a silicon compound and titanosilicate after used in an ammoximation reaction” may be referred to as simply a “fired product of a solid containing a silicon compound”). For example, when ammoximation is performed in a batch manner, at least a part of a mixture of a solid containing a silicon compound and titanosilicate retrieved after an ammoximation reaction is removed every one or a few batch and a firing product of a solid containing a silicon compound and titanosilicate may be supplemented. When the ammoximation reaction is performed in a continuous manner, a part of a mixture of a solid containing a silicon compound and titanosilicate used in the ammoximation reaction may be removed from the reaction system at an appropriate interval during operation and a firing product of a solid containing a silicon compound and titanosilicate may be supplemented, or at least a part of a mixture of a solid containing a silicon compound and titanosilicate retrieved after the ammoximation reaction may be removed when operation is stopped and a firing product of a solid containing a silicon compound and titanosilicate may be supplemented. The mixture of a solid containing a silicon compound and titanosilicate retrieved after the ammoximation reaction is reused as a firing product of a solid containing a silicon compound and titanosilicate after firing. Optionally, together with supplement of a firing product of a solid containing a silicon compound and titanosilicate in an ammoximation reaction, a solid containing a silicon compound which has not been used in an ammoximation reaction and/or fresh titanosilicate may be supplemented. The amount of the solid containing a silicon compound which has not been used in an ammoximation reaction to be supplemented is preferably an amount equivalent to a Si amount which is excreted outside the reaction system during the ammoximation reaction. In a continuous ammoximation reaction, the Si amount excreted outside the reaction system may be obtained by measuring the Si concentration in the retrieved reaction mixture based on the amount of the retrieved reaction mixture. The amount of the fresh titanosilicate to be supplemented may be adequately determined depending on the catalytic activity yet declined even when the reaction is being performed as above.

A known method may be appropriately applied for post-treatment operation of the reaction mixture obtained through an ammoximation reaction and, for example, by distilling a liquid phase of the reaction mixture, remaining unreacted ammonia and a solvent which is used, if required, are separated and retrieved as a fraction and a bottom product containing remaining unreacted cyclohexanone and cyclohexanone oxime may be obtained. Then, by extracting remaining unreacted cyclohexanone and cyclohexanone oxime with an organic solvent from this bottom product and by distilling the extract, if required, after washing and concentrating it, if required, to separate and retrieve unreacted cyclohexanone and the organic solvent used for extraction as respective fractions, purified cyclohexanone oxime may be obtained. The retrieved ammonia, solvent, cyclohexanone and the organic solvent used for extraction may be reused. Further, cyclohexanone oxime thus obtained may be subjected to a Beckmann rearrangement reaction in a liquid or gas phase to produce ε-caprolactam.

### EXAMPLES

Examples of the present invention are shown below. However, the present invention is not limited to them. Analyses of carbon contents and nitrogen contents in catalysts used in Beckmann rearrangement reactions were performed on a quantitative analyzing instrument [SUMIGRAPH NCH-21, Sumika Chemical Analysis Service, Ltd. (Oxygen recirculating combustion method/TCD-GC detection method)]. A space velocity WHSV (h⁻¹) of cyclohexanone oxime in a Beckmann rearrangement reaction was calculated by dividing a feed rate (g/hour) of cyclohexanone oxime by an amount (g) of a Beckmann rearrangement reaction catalyst. Analyses of liquid phases in production of cyclohexanone oxime were performed by gas chromatography. Further, catalyst lifetimes were judged according to oxygen concentrations in autoclaves. When a catalytic activity declines, an amount of oxygen generated by thermal decomposition of hydrogen peroxide increases to raise an oxygen concentration in a system rapidly. Therefore, it is considered that the longer a period from onset of operation to a time when the longer the oxygen concentration rapidly increases is, the longer the catalytic lifetime is.

### Referential Example 1

<Preparation of Catalysts (A) and (B) used in a Beckmann Rearrangement Reaction>

A particle mainly comprising MFI zeolite of crystalline silica, whose particle size was 0.3 mm or smaller, were used as a catalyst. A Beckmann rearrangement reaction was performed at 380° C. for 6 months by removing reaction generation gas with supplying vaporized cyclohexanone oxime, vaporized methanol and a nitrogen gas into a fluidized-bed reactor in which this catalyst flowed. During this, the space velocity WHSV of cyclohexanone oxime was set to 2 h⁻¹. The supplying proportion of methanol was set to 1.8 kg with respect to 1 kg of cyclohexanone oxime and the supplying proportion of nitrogen gas was set to 0.8 L with respect to 1 kg of cyclohexanone oxime. Further, during this, by removing a part of the catalyst from the reactor and introducing it into a firing furnace to fire it at 430° C. for a retention time of 20 hours with an air flow, and by introducing it into the firing furnace again, the catalyst was circulated between the reactor and the firing furnace. After the Beckmann rearrangement reaction, a part of the catalyst from the firing furnace was removed to be a catalyst used in a Beckmann rearrangement reaction (A). This catalyst used in a Beckmann rearrangement reaction (A) had the carbon content of 0.13 weight % and the nitrogen content of 0.006 weight %. A part of the catalyst from the reactor was removed to be a catalyst used in a Beckmann rearrangement reaction (B). This catalyst used in a Beckmann rearrangement reaction (B) had a carbon content of 1.45 weight % and a nitrogen content of 0.11 weight %.

### Example 1

### <Production of Cyclohexanone Oxime>

To a 300-mL volume of autoclave equipped with a stirrer, an inner surface of which is lined with a fluorine resin, were placed 1.5 g of titanosilicate (TS-1) and 1.5 g of the catalyst (A) used in a Beckmann rearrangement reaction obtained in Referential Example 1. After adding 100 mL of hydrous t-butyl alcohol (water, 15 weight %), stirring was initiate at 390 rpm. With continuously introducing cyclohexanone at a rate of 19.6 g/hour, hydrous t-butyl alcohol (water 15 weight %) at a rate of 34.8 g/hour, ammonia at a rate of 6.45 g/hour (1.9 mole equivalent to cyclohexanone), and 60 weight % of a hydrogen peroxide aqueous solution at a rate of 13.0 g/hour (1.15 mole equivalent to cyclohexanone) to this, the liquid phase of the reaction mixture was removed a sintered metal filter of stainless steel so that the volume of the reaction mixture in the autoclave was maintained at 100 mL to perform a continuous ammoximation reaction. During this, the reaction temperature was maintained at 95° C. and the reaction pressure was maintained at 0.35 MPa (as an absolute pressure) by pressurizing with helium gas. Further, with flowing helium gas at a rate of 1.2 L/hour in the gas phase in the reactor, the oxygen concentration was monitored as an index of catalyst deterioration. After 5.5 hours from an onset of operation (onset of retrieve of a liquid phase), the liquid phase of the reaction mixture from an autoclave was analyzed on a gas chromatography. As a result, the cyclohexanone conversion was 99.5% and the cyclohexanone oxime selectivity was 99.4%. Since at a time when 260 hours passed from the onset of operation, the oxygen concentration in the exhaust gas exceeded 10 volume %, the operation was stopped.

### Example 2

An ammoximation reaction was performed according to Example 1 <Production of Cyclohexanone Oxime> with an exception that the amount of the catalyst (A) used in a

Beckmann rearrangement reaction was 3.0 g. After 5.5 hours from an onset of operation (onset of retrieve of a liquid phase), the liquid phase of the reaction mixture from an autoclave was analyzed on a gas chromatography. As a result, the cyclohexanone conversion was 99.5% and the cyclohexanone oxime selectivity was 99.7%. Since at a time when 339 hours passed from the onset of operation, the oxygen concentration in the exhaust gas exceeded 10 volume %, the operation was stopped.

### Example 3

An ammoximation reaction was performed according to Example 1 <Production of Cyclohexanone Oxime> with an exception that the amount of the catalyst (A) used in a Beckmann rearrangement reaction was 8.0 g. After 5.5 hours from an onset of operation (onset of retrieve of a liquid phase), the liquid phase of the reaction mixture from an autoclave was analyzed on a gas chromatography. As a result, the cyclohexanone conversion was 99.5% and the cyclohexanone oxime selectivity was 99.7%. Since at a time when 434 hours passed from the onset of operation, the oxygen concentration in the exhaust gas exceeded 10 volume %, the operation was stopped.

### Example 4

An ammoximation reaction was performed according to Example 1 <Production of Cyclohexanone Oxime> with an exception that 1.5 g of the catalyst (A) used in a Beckmann rearrangement reaction was replaced with 1.5 g of Catalyst (B) used in a Beckmann rearrangement reaction obtained in Referential Example 1. After 5.5 hours from an onset of operation (onset of retrieve of a liquid phase), the liquid phase of the reaction mixture from an autoclave was analyzed on a gas chromatography. As a result, the cyclohexanone conversion was 99.4% and the cyclohexanone oxime selectivity was 99.6%. Since at a time when 240 hours passed from the onset of operation, the oxygen concentration in the exhaust gas exceeded 10 volume %, the operation was stopped.

### Comparative Example 1

An ammoximation reaction was performed according to Example 1 <Production of Cyclohexanone Oxime> with an exception that the catalyst (A) used in a Beckmann rearrangement reaction was not used. After 5.5 hours from an onset of operation (onset of retrieve of a liquid phase), the liquid phase of the reaction mixture from an autoclave was analyzed on a gas chromatography. As a result, the cyclohexanone conversion was 99.2% and the cyclohexanone oxime selectivity was 99.5%. Since at a time when 144 hours passed from the onset of operation, the oxygen concentration in the exhaust gas exceeded 10 volume %, the operation was stopped.

### Comparative Example 2

An ammoximation reaction was performed according to Example 1 <Production of Cyclohexanone Oxime> with an exception that 1.5 g of the catalyst (A) used in a Beckmann rearrangement reaction was replaced with 8.0 g of silica gel [Wakogel® B-0, Wako Pure Chemical Industries, Ltd.]. After 5.5 hours from an onset of operation (onset of retrieve of a liquid phase), the liquid phase of the reaction mixture from an autoclave was analyzed on a gas chromatography. As a result, the cyclohexanone conversion was 99.1% and the cyclohexanone oxime selectivity was 99.5%. Since at a time when 290 hours passed from the onset of operation, the oxygen concentration in the exhaust gas exceeded 10 volume %, the operation was stopped.

1. A method for manufacturing cyclohexanone oxime comprising
performing an ammoximation reaction of cyclohexanone with hydrogen peroxide and ammonia in the presence of titanosilicate and a solid containing a silicon compound, wherein the solid containing a silicon compound is one that had been used as a catalyst in a Beckmann rearrangement reaction of cyclohexanone oxime.

2. The method according to claim 1, wherein the silicon compound is at least one selected from a group consisting of zeolite, silica alumina, a complex oxide of silica and a metal oxide other than silica, and amorphous silica.

3. The method according to claim 1 or 2, wherein the solid further contains cokes.

4. The method according to claim 3, wherein a carbon content is 5.0 weight % or lower in the solid.

5. The method according to claim 3, wherein a carbon content is 5.0 weight % or lower and a nitrogen content is 0.50 weight % or lower in the solid.
